(19) United States
(12) Patent Application Publication
(10) Pub. No.: US 2026/0232629 A1
(43) Pub. Date: Aug. 13, 2026

(54) Covalent Degraders of Oncogenic Transcription Factors

(71) Applicant: The Regents of the University of California, Oakland, CA (US)

(72) Inventors: Daniel K. Nomura, Berkeley, CA (US); Flor Gowans, Berkeley, CA (US); Nafsika Forte, Berkeley, CA (US)

(73) Assignee: The Regents of the University of California, Oakland, CA (US)

(21) Appl. No.: 19/637,860

(22) Filed: Apr. 2, 2026

Related U.S. Application Data
(63) Continuation of application No. PCT/US24/52465, filed on Oct. 23, 2024.
(60) Provisional application No. 63/593,090, filed on Oct. 25, 2023.

(51) Int. Cl. A61K 31/40; A61P 35/00; C07D 207/16; C07K 14/705

(52) U.S. Cl. CPC A61K 31/40; A61P 35/00; C07D 207/16; C07K 14/705

(57) ABSTRACT

Covalent degraders of oncogenic transcription factors are formulated as pharmaceutical compositions for therapeutic use.

### CROSS-REFERENCES TO RELATED APPLICATIONS

This application is a continuation of PCT/US24/52465, filed: Oct. 23, 2024, which claims priority to U.S. Provisional Application No. 63/593,090, filed: Oct. 25, 2023, the disclosures of which are hereby incorporated by reference in its entirety for all purposes.

### GOVERNMENT SUPPORT CLAUSE

This invention was made with government support under grant CA263814 awarded by the National Institutes of Health. The government has certain rights in the invention.

### INTRODUCTION

B-catenin (CTNNB1) is an oncogenic transcription factor that drives the pathogenesis of many different types of human cancers, including liver, lung, colorectal, breast, and ovarian cancers¹⁻³. CTNNB1 regulates cell-cell adhesion as part of a larger protein complex with E-cadherin and β-catenin. CTNNB1 is also a transcription factor that is activated by upstream Wnt signaling pathways that regulates genes involved in cell proliferation, survival, epithelial-to-mesenchymal transition, migration, and metastasis¹⁻³. CTNNB1 is highly regulated by the ubiquitin-proteasome system, wherein the E3 ubiquitin ligase β-TrCP1 recognizes N-terminus of CTNNB1 upon phosphorylation by GSK3α and GSK3β¹⁻³. Mutations in CTNNB1 are commonly found in a variety of cancers that are often located in the N-terminal segment that is recognized by E3 ligases preventing ubiquitination and degradation of CTNNB1, thereby enhancing CTNNB1 oncogenic transcriptional activity¹⁻³. Mutations have also been found in the Wnt pathway, including in APC and axin that act to enhance CTNNB1 levels and activity¹⁻³.

Many efforts have been made to target the Wnt pathway for cancer therapy. PKF115-584 and CGP04090 disrupt interactions between CTNNB1 and TCF⁴⁻⁵. ICG-001 disrupts binding between CTNNB1 and CBP⁶. Tankyrase inhibitors such as IWR-1 and XAV939 to stabilize Axin and the CK1 activator Pryivinium to enhance the activity of the destruction complex have been discovered to enhance the degradation of CTNNB1⁷⁻⁸. Direct CTNNB1 binding degraders such as methyl 3-{[(4-methylphenyl)sulfonyl]amino}benzoate) or MSAB that attenuate colorectal tumor growth have also been discovered. However, given the rapid turnover rate of CTNNB1; more durable pharmacological strategies are needed to directly and fully target, degrade, and inhibit the activity of CTNNB1 for cancer therapy⁹. Direct targeting of oncogenic transcription factors like CTNNB1 has also remained challenging due to its structural flexibility, intrinsic disorder, and lack of ligandable hotspots².

There has been a recent surge in covalent drug discovery because of the ability of covalent drugs to access classically undruggable targets as well as shallower binding pockets through a combination of reactivity and affinity-driven mechanisms. With advances in covalent chemoproteomic strategies such as activity-based protein profiling (ABPP), the overall target engagement and proteome-wide selectivity of these compounds can also be assessed and coupled with medicinal chemistry efforts to not only optimize potency, but also specificity, to eventually reduce off-target toxicological liabilities. Previously, covalent chemoproteomic approaches were also used to covalently target an intrinsically disordered cysteine within MYC to destabilize and degrade MYC leading to antitumorigenic effects.

### SUMMARY OF THE INVENTION

The invention provides covalent degraders of oncogenic transcription factors, related compositions and methods.

In an aspects and embodiments the invention provides:
1. A compound of formula:

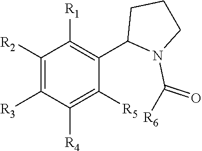

wherein
a) one of R1-R5 is methoxy, and the others are independently selected from H, halide, hydroxyl, C1-C4 alkyl, C1-C4 fluorinated alkyl, C1-C4 alkyloxy, C1-C4 fluorinated alkyloxy, or other substituents described herein, wherein two of the other R1-R5 may be joined in a 5 or 6 membered ring;
b) NCOR6 is a amide-linked cysteine-reactive covalent ligand warhead,
including a pharmaceutically-acceptable salt thereof, and excluding EN83.
2. A compound herein wherein R2, R3 and R5 are H.
3. A compound herein wherein one of R1 and R4 is CHF₃.
4. A compound herein wherein R6 is CH₂Cl.
5. A compound herein wherein the warhead is disclosed in:
a) Boike, et al. Advances in covalent drug discovery. Nat Rev Drug Discov 21, 881-898 (2022), including EGFR inhibitors (such as gefitinib, erlotinib, fatinib, neratinib, dacomitinib, WZ4002, osimertinib, rociletinib), BTK inhibitors (such as ibrutinib, acalabrutinib, zanubrutinib), Janus kinase 3 (JAK3) inhibitors (such as ritlecitinib), fibroblast growth factor receptor 4 (FGFR4) inhibitors (such as fisogatinib, roblitinib), KRAS (G12C) inhibitors (such as sotorasib, adagrasib, JNJ-7469915), SARS-CoV-2 main protease inhibitors (such as nirmatrelvir, rupintrivir), proteasome inhibitors (such as bortezomib, carfilzomib, ixazomib, oprozomib), other boron-containing drugs (such as tavaborole, crisaborole, vaborbactam) and mutant-haemoglobin modulators (such as voxelotor);
b) Grabrijan et al., Covalent inhibitors of bacterial peptidoglycan biosynthesis enzyme MurA with chloroacetamide warhead Eur J Med Chem, 2022 Dec. 5:243:114752, see Table 2;
c) Huang, et al., Covalent Warheads Targeting Cysteine Residue: The Promising Approach in Drug Development. Molecules 2022, 27,7728 or
d) Petri, et al., An electrophilic warhead library for mapping the reactivity and accessibility of tractable cysteines in protein kinases, Eu J Med Chem, 207, Dec. 1, 2020, 112836.
5. A compound wherein CORE is but-2-en-1-one or but-2-ene-1,4-dione linked phenyl or phenyl substituted with 1-5 substituents selected from: halide, hydroxyl, C1-C4 alkyl, C1-C4 fluorinated alkyl, C1-C4 alkyloxy, C1-C4 fluorinated alkyloxy, NRaRb, NHCORa, or other substituents described herein, wherein Ra and Rb are independently H, C1-C4 alkyl, C1-C4 fluorinated alkyl, C1-C4 alkyloxy, C1-C4 fluorinated alkyloxy, or other substituents described herein.
6. A compound herein of formula:

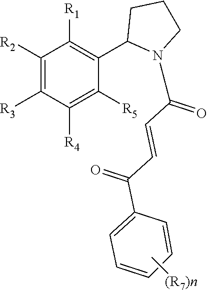

wherein
a) one of R1-R5 is methoxy, and the others are independently selected from H, halide, hydroxyl, C1-C4 alkyl, C1-C4 fluorinated alkyl, C1-C4 alkyloxy, C1-C4 fluorinated alkyloxy, or other substituents described herein, wherein two of the other R1-R5 may be joined in a 5 or 6 membered ring,
b) R7 is selected from: H, halide, hydroxyl, C1-C4 alkyl, C1-C4 fluorinated alkyl, C1-C4 alkyloxy, C1-C4 fluorinated alkyloxy, NRaRb, NHCORa, or other substituents described herein, wherein Ra and Rb are independently H, C1-C4 alkyl, C1-C4 fluorinated alkyl, C1-C4 alkyloxy, C1-C4 fluorinated alkyloxy, or other substituents described herein,
c) n is 1-5.
7. A compound herein, such as NF compounds of Table 1.
8. A protein comprising a cysteine covalently modified with a compound herein.
9. A protein herein comprising an oncogenic transcription factor, such as: β-catenin/Wnt, NF-κB, AP1. STAT3, HIF-1, MYC, ETS1, E2F.
10. A protein comprising a modified β-catenin (CTNNB1) comprising a covalent modification of C439, C466, C520 or C619.
11. A pharmaceutical composition comprising a compound herein and a pharmaceutically-acceptable excipient, in unit dosage or packaging.
12. A method of modifying a protein, comprising contacting the protein with a compound herein under conditions wherein a cysteine residue of the protein is covalently modified by the compound.
13. A method herein wherein the protein is selected from Wnt/β-catenin (CTNNB1), β-NF-κB, AP1. STAT3, HIF-1, MYC, ETS1, E2F.
14. A method of modifying a protein comprising a β-catenin (CTNNB1), comprising covalently modifying C439, C466, C520 or C619.
15. A therapeutic method comprising administering to a person in need thereof a compound herein, such as using methods described or cited in Vishnoi et al., Transcription Factors in Cancer Development and Therapy, Cancers (Basel). 2020 August; 12(8): 2296.
16. A therapeutic method herein to treat a cancer in the person, such as: breast cancer, prostate cancer, colon cancer, multiple myeloma, liver cancer, ovarian cancer, lung cancer, acute myeloid leukemia, renal cancer, hepatocellular carcinoma, melanoma, colorectal cancer, bladder cancer, lung adenocarcinoma, pancreatic cancer, glioblastoma or anaplastic glioma, small-cell lung cancer, or other cancers associated with an oncogenic transcription factor.
17. A therapeutic method herein to inhibit an oncogenic transcription factor in the person, such as: β-catenin/Wnt, NF-κB, AP1. STAT3, HIF-1, MYC, ETS1, E2F.

The invention encompasses all combinations of the particular embodiments recited herein, as if each combination had been laboriously recited.

### Description of Particular Embodiments of the Invention

Unless contraindicated or noted otherwise, in these descriptions and throughout this specification, the terms “a” and “an” mean one or more, the term “or” means and/or. It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

The term hydrocarbyl refers to hydrocarbon radical, including alkyl, alkenyl, alkynyl and aryl.

The term “alkyl” refers to a hydrocarbon group selected from linear and branched saturated hydrocarbon groups of 1-18, or 1-12, or 1-6 carbon atoms. Examples of the alkyl group include methyl, ethyl, l-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), and 1,1-dimethylethyl or t-butyl (“t-Bu”). Other examples of the alkyl group include 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups.

Lower alkyl means 1-8, preferably 1-6, more preferably 1-4 carbon atoms; lower alkenyl or alkynyl means 2-8, 2-6 or 2-4 carbon atoms.

The term “alkenyl” refers to a hydrocarbon group selected from linear and branched hydrocarbon groups comprising at least one C═C double bond and of 2-18, or 2-12, or 2-6 carbon atoms. Examples of the alkenyl group may be selected from ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl, and hexa-1,3-dienyl groups.

The term “alkynyl” refers to a hydrocarbon group selected from linear and branched hydrocarbon group, comprising at least one C═C triple bond and of 2-18, or 2-12, or 2-6 carbon atoms. Examples of the alkynyl group include ethynyl, 1-propynyl, 2-propynyl (propargyl), 1-butynyl, 2-butynyl, and 3-butynyl groups.

The term “cycloalkyl” refers to a hydrocarbon group selected from saturated and partially unsaturated cyclic hydrocarbon groups, comprising monocyclic and polycyclic (e.g., bicyclic and tricyclic) groups. For example, the cycloalkyl group may be of 3-12, or 3-8, or 3-6 carbon atoms. Even further for example, the cycloalkyl group may be a monocyclic group of 3-12, or 3-8, or 3-6 carbon atoms. Examples of the monocyclic cycloalkyl group include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and cyclododecyl groups. Examples of the bicyclic cycloalkyl groups include those having 7-12 ring atoms arranged as a bicycle ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. The ring may be saturated or have at least one double bond (i.e. partially unsaturated), but is not fully conjugated, and is not aromatic, as aromatic is defined herein.

The term “aryl” herein refers to a group selected from: 5- and 6-membered carbocyclic aromatic rings, for example, phenyl; bicyclic ring systems such as 7-12 membered bicyclic ring systems wherein at least one ring is carbocyclic and aromatic, selected, for example, from naphthalene, indane, and 1,2,3,4-tetrahydroquinoline; and tricyclic ring systems such as 10-15 membered tricyclic ring systems wherein at least one ring is carbocyclic and aromatic, for example, fluorene.

For example, the aryl group is selected from 5- and 6-membered carbocyclic aromatic rings fused to a 5- to 7-membered cycloalkyl or heterocyclic ring optionally comprising at least one heteroatom selected from N, O, and S, provided that the point of attachment is at the carbocyclic aromatic ring when the carbocyclic aromatic ring is fused with a heterocyclic ring, and the point of attachment can be at the carbocyclic aromatic ring or at the cycloalkyl group when the carbocyclic aromatic ring is fused with a cycloalkyl group. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Aryl, however, does not encompass or overlap with heteroaryl, separately defined below. Hence, if one or more carbocyclic aromatic rings are fused with a heterocyclic aromatic ring, the resulting ring system is heteroaryl, not aryl, as defined herein.

The term “halogen” or “halo” refers to F, Cl, Br or I.

The term “heteroalkyl” refers to alkyl comprising at least one heteroatom.

The term “heteroaryl” refers to a group selected from:
5- to 7-membered aromatic, monocyclic rings comprising 1, 2, 3 or 4 heteroatoms selected from N, O, and S, with the remaining ring atoms being carbon;
8- to 12-membered bicyclic rings comprising 1, 2, 3 or 4 heteroatoms, selected from N, O, and S, with the remaining ring atoms being carbon and wherein at least one ring is aromatic and at least one heteroatom is present in the aromatic ring; and
11- to 14-membered tricyclic rings comprising 1, 2, 3 or 4 heteroatoms, selected from N, O, and S, with the remaining ring atoms being carbon and wherein at least one ring is aromatic and at least one heteroatom is present in an aromatic ring.

For example, the heteroaryl group includes a 5- to 7-membered heterocyclic aromatic ring fused to a 5- to 7-membered cycloalkyl ring. For such fused, bicyclic heteroaryl ring systems wherein only one of the rings comprises at least one heteroatom, the point of attachment may be at the heteroaromatic ring or at the cycloalkyl ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1.

Examples of the heteroaryl group include, but are not limited to, (as numbered from the linkage position assigned priority 1) pyridyl (such as 2-pyridyl, 3-pyridyl, or 4-pyridyl), cinnolinyl, pyrazinyl, 2,4-pyrimidinyl, 3,5-pyrimidinyl, 2,4-imidazolyl, imidazopyridinyl, isoxazolyl, oxazolyl, thiazolyl, isothiazolyl, thiadiazolyl, tetrazolyl, thienyl, triazinyl, benzothienyl, furyl, benzofuryl, benzoimidazolyl, indolyl, isoindolyl, indolinyl, phthalazinyl, pyrazinyl, pyridazinyl, pyrrolyl, triazolyl, quinolinyl, isoquinolinyl, pyrazolyl, pyrrolopyridinyl (such as 1H-pyrrolo[2,3-b]pyridin-5-yl), pyrazolopyridinyl (such as 1H-pyrazolo[3,4-b]pyridin-5-yl), benzoxazolyl (such as benzo[d]oxazol-6-yl), pteridinyl, purinyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, furazanyl, benzofurazanyl, benzothiophenyl, benzothiazolyl, benzoxazolyl, quinazolinyl, quinoxalinyl, naphthyridinyl, furopyridinyl, benzothiazolyl (such as benzo[d]thiazol-6-yl), indazolyl (such as 1H-indazol-5-yl) and 5,6,7,8-tetrahydroisoquinoline.

The term “heterocyclic” or “heterocycle” or “heterocyclyl” refers to a ring selected from 4- to 12-membered monocyclic, bicyclic and tricyclic, saturated and partially unsaturated rings comprising at least one carbon atoms in addition to 1, 2, 3 or 4 heteroatoms, selected from oxygen, sulfur, and nitrogen. “Heterocycle” also refers to a 5- to 7-membered heterocyclic ring comprising at least one heteroatom selected from N, O, and S fused with 5-, 6-, and/or 7-membered cycloalkyl, carbocyclic aromatic or heteroaromatic ring, provided that the point of attachment is at the heterocyclic ring when the heterocyclic ring is fused with a carbocyclic aromatic or a heteroaromatic ring, and that the point of attachment can be at the cycloalkyl or heterocyclic ring when the heterocyclic ring is fused with cycloalkyl.

“Heterocycle” also refers to an aliphatic spirocyclic ring comprising at least one heteroatom selected from N, O, and S, provided that the point of attachment is at the heterocyclic ring. The rings may be saturated or have at least one double bond (i.e. partially unsaturated). The heterocycle may be substituted with oxo. The point of the attachment may be carbon or heteroatom in the heterocyclic ring. A heterocyle is not a heteroaryl as defined herein.

Examples of the heterocycle include, but not limited to, (as numbered from the linkage position assigned priority 1) 1-pyrrolidinyl, 2-pyrrolidinyl, 2,4-imidazolidinyl, 2,3-pyrazolidinyl, 1-piperidinyl, 2-piperidinyl, 3-piperidinyl, 4-piperidinyl, 2,5-piperazinyl, pyranyl, 2-morpholinyl, 3-morpholinyl, oxiranyl, aziridinyl, thiiranyl, azetidinyl, oxetanyl, thietanyl, 1,2-dithietanyl, 1,3-dithietanyl, dihydropyridinyl, tetrahydropyridinyl, thiomorpholinyl, thioxanyl, piperazinyl, homopiperazinyl, homopiperidinyl, azepanyl, oxepanyl, thiepanyl, 1,4-oxathianyl, 1,4-dioxepanyl, 1,4-oxathiepanyl, 1,4-oxaazepanyl, 1,4-dithiepanyl, 1,4-thiazepanyl and 1,4-diazepane 1,4-dithianyl, 1,4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1,4-dioxanyl, 1,3-dioxolanyl, pyrazolinyl, pyrazolidinyl, dithianyl, dithiolanyl, pyrazolidinylimidazolinyl, pyrimidinonyl, 1,1-dioxo-thiomorpholinyl, 3-azabicyco[3.1.0]hexanyl, 3-azabicyclo[4.1.0]heptanyl and azabicyclo[2.2.2]hexanyl. Substituted heterocycle also includes ring systems substituted with one or more oxo moieties, such as piperidinyl N-oxide, morpholinyl-N-oxide, 1-oxo-1-thiomorpholinyl and 1, 1-dioxo-1-thiomorpholinyl.

Substituents are selected from: halogen, —R′, —OR′, ═O, ═NR′, ═N—OR′, —NR′R″, —SR′, —SiR′R″R″′, —OC(O)R′, —C(O)R′, —CO₂R′, —CONR′R″, —OC(O)NR′R″, —NR″C(O)R′, —NR′—C(O)NR″R″′, —NR′—SO₂NR″′, —NR″CO₂R′, —NH—C(NH₂)═NH, —NR′C(NH₂)═NH, —NH—C(NH₂)═NR′, —S(O)R′, —SO₂R′, —SO₂NR′R″, —NR″SO₂R, —CN and —NO₂, —N₃, —CH(Ph)₂, perfluoro(C1-C4)alkoxy and perfluoro(C1-C4)alkyl, in a number ranging from zero to three, with those groups having zero, one or two substituents being particularly preferred. R, R′, R″ and R″ each independently refer to hydrogen, unsubstituted (C1-C8)alkyl and heteroalkyl, unsubstituted aryl, aryl substituted with one to three halogens, unsubstituted alkyl, alkoxy or thioalkoxy groups, or aryl-(C1-C4)alkyl groups. When R′ and R″ are attached to the same nitrogen atom, they can be combined with the nitrogen atom to form a 5-, 6- or 7-membered ring. Hence, —NR′R″ includes 1-pyrrolidinyl and 4-morpholinyl, “alkyl” includes groups such as trihaloalkyl (e.g., —CF₃ and —CH₂CF₃), and when the aryl group is 1,2,3,4-tetrahydronaphthalene, it may be substituted with a substituted or unsubstituted (C3-C7)spirocycloalkyl group. The (C3-C7)spirocycloalkyl group may be substituted in the same manner as defined herein for “cycloalkyl”.

Preferred substituents are selected from: halogen, —R′, —OR′, ═O, —NR′R″, —SR′, —SiR′R″R″′, —OC(O)R′, —C(O)R′, —CO₂R′, —CONR′R″, —OC(O)NR′R″, —NR″C(O)R′, —NR″CO₂R′, —NR′—SO₂NR″R″′, —S(O)R′, —SO₂R′, —SO₂NR′R″, —NR″SO₂R, —CN and —NO₂, perfluoro(C1-C4)alkoxy and perfluoro(C1-C4)alkyl, where R′ and R″ are as defined above.

The term “fused ring” refers to a polycyclic ring system, e.g., a bicyclic or tricyclic ring system, in which two rings share only two ring atoms and one bond in common. Examples of fused rings may comprise a fused bicyclic cycloalkyl ring such as those having from 7 to 12 ring atoms arranged as a bicyclic ring selected from [4,4], [4,5], [5,5], [5,6] and [6,6] ring systems as mentioned above; a fused bicyclic aryl ring such as 7 to 12 membered bicyclic aryl ring systems as mentioned above, a fused tricyclic aryl ring such as 10 to 15 membered tricyclic aryl ring systems mentioned above; a fused bicyclic heteroaryl ring such as 8- to 12-membered bicyclic heteroaryl rings as mentioned above, a fused tricyclic heteroaryl ring such as 11- to 14-membered tricyclic heteroaryl rings as mentioned above; and a fused bicyclic or tricyclic heterocyclyl ring as mentioned above.

The compounds may contain an asymmetric center and may thus exist as enantiomers. Where the compounds possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds and/or pharmaceutically acceptable salts thereof are intended to be included. Unless specifically mentioned otherwise, reference to one isomer applies to any of the possible isomers. Whenever the isomeric composition is unspecified, all possible isomers are included.

The compounds of the invention may also contain unnatural proportions of atomic isotopes at one or more of the atoms that constitute such compounds, such as deuterium, e.g. —CD₃, CD₂H or CDH₂ in place of methyl. For example, the compounds may be radiolabeled with radioactive isotopes, such as for example tritium (³H), iodine-125 (¹²⁵I) or carbon-14 (¹⁴C). All isotopic variations of the compounds of the invention, whether radioactive or not, are intended to be encompassed within the scope of the invention.

### EXAMPLES

### Covalent Degrader of the Oncogenic Transcription Factor β-Catenin

In this example we show that EN83 directly and covalently targets CTNNB1 through targeting four distinct cysteines within the armadillo repeat domain—C439, C466, C520, and C619—leading to a destabilization of CTNNB1. Using covalent chemoproteomic approaches, we show that EN83 directly engages CTNNB1 in cells with a moderate degree of selectivity. We further demonstrate that direct covalent targeting of three of these four cysteines—C466, C520, and C619—in cells contributes to CTNNB1 degradation in cells. We also demonstrate that EN83 can be further optimized to yield more potent CTNNB1 binders and degraders. Our results show that covalent chemoproteomic approaches can be used to covalent target and degrade challenging transcription factors like CTNNB1 through a destabilization-mediated degradation.

We screened a cysteine-reactive covalent ligand library to develop covalent monovalent degraders of CTNNB1 that eliminate CTNNB1 in a ubiquitin-proteasome dependent manner through targeting cysteines on CTNNB1.

### Discovery of a Covalent Degrader of CTNNB1

To identify a covalent degrader of CTNNB1, we screened a library of 2100 cysteine-reactive covalent ligands consisting of acrylamides and chloroacetamides in HEK293 cells expressing CTNNB1 with a N-terminal HiBiT tag incorporated into the endogenous loci of CTNNB1. We identified 17 hits lowered CTNNB1 levels. We subsequently eliminated hits that had appeared in previous screens run in our lab and counter screened any resulting hits to identify compounds that showed attenuation in CTNNB1 loss upon pre-incubation with either a proteasome inhibitor bortezomib or the Cullin E3 ligase NEDDylation inhibitor MLN4924. We identified EN83 as the only hit that showed dose-responsive CTNNB1 loss that was significantly attenuated by proteasome or NEDDylation inhibition.

We further confirmed proteasome-mediated loss of CTNNB1 by Western blotting in HEK293T cells. We also demonstrated loss of CTNNB1 in colorectal cancer cells that are driven by CTNNB1, including HT29, COLO, and SW480 cancer cell lines. While we observed CTNNB1 loss in the HiBiT-tagged CTNNB1-expressing HEK293T cells at 24 h, we only observed CTNNB1 loss in HT29, COLO, and SW480 cells at earlier acute timepoints of 2 and 4 h, but CTNNB1 levels had recovered by 24 h in these cancer cell lines. This may be due to more rapid turn-over rates of CTNNB1 in more cancer-relevant cell lines.

### Characterization of EN83 as a Direct CTNNB1 Binder

We next sought to determine whether EN83 directly and covalently bound to CTNNB1. We found that EN83 dose-responsively displaced cysteine-reactive fluorescent probe labeling of pure human CTNNB1 protein by gel-based ABPP. We further performed tandem mass spectrometry analysis (MS/MS) on tryptic digests from CTNNB1 pure protein incubated with EN83 and found four cysteines that were modified by EN83—C439, C466, C520, C619. Each of these cysteines reside in armadillo repeat motifs within the CTNNB1 structure suggesting that these ligands may be binding across several structurally similar domains. To further confirm direct covalent binding of EN83 to CTNNB1, we synthesized an alkyne-functionalized analog of EN83, NF686 and showed direct covalent dose-responsive probe labeling of pure CTNNB1 protein by NF686. We further demonstrated that the covalency is necessary since a non-reactive analog of EN83, NF602, does not show binding to CTNNB1 and does not alter CTNNB1 levels by HiBiT or Western blotting detection. Our results thus compelling demonstrated that EN83 directly binds to CTNNB1.

We next wanted to confirm that EN83 engages CTNNB1 in cells. We first used the alkyne probe NF686 in cells to demonstrate that CTNNB1 could be enriched from cells with the probes without enriching unrelated targets such as GAPDH. We next performed a mass spectrometry-based ABPP or isodesthiobiotin-ABPP (isoDTB-ABPP) to map overall proteome-wide cysteine-reactivity of EN83 in cells using previously established methods. While EN83 is only moderately selective, we did observe that EN83 significantly engaged C619 on CTNNB1 alongside 139 other targets significantly engaged by over 50% among 8251 cysteines detected and quantified across three biological replicates. Among the off-targets of EN83, none of the targets were within the Wnt signaling pathway, including AXIN1, BTRC, or GSK3A or GSK3B. Cellular thermal shift assay from EN83 cellular treatment showed significant thermal destabilization of CTNNB1 in cells, indicating that the degradation of CTNNB1 may be caused by direct covalent targeting of CTNNB1 leading to destabilization of CTNNB1 folding and subsequent ubiquitination and degradation.

We next sought to confirm the contributions of the cysteines targeted by EN83 in CTNNB1 to CTNNB1 degradation. We expressed either FLAG-tagged wild-type, C439S, C466S, C520S, or C619S mutant CTNNB1 in cells and showed that mutation of C466, C520, or C619, but not C439, to serines significantly attenuated CTNNB1 degradation in cells. These results demonstrated that direct targeting of three out of the four cysteines identified within CTNNB1 led to the degradation of CTNNB1.

### Improving Potency of EN83 Against CTNNB1

Towards improving potency of EN83 against CTNNB1 and exploring structure-activity relationships, we synthesized several analogs of EN83. EN83 is a racemic mixture of two enantiomers. We first separated the individual enantiomers to determine whether there was a particular enantiomer that was more potent. Both enantiomers of EN83 showed comparable lowering of CTNNB1 levels and showed equivalent CTNNB1 binding, indicating that there was no difference between the two enantiomers. We found that replacement of the methoxy groups with trifluoromethyl moieties, NF740 and NF741, was tolerated for both binding to CTNNB1 by gel-based ABPP and lowering CTNNB1 HiBiT levels in HEK293 cells in a dose-responsive manner. This lowering of CTNNB1 was also confirmed to be proteasome-dependent by Western blotting. We also sought to replace the chloroacetamide cysteine-reactive warhead for further development. Replacement of the chloroacetamide warhead with an oxo-phenylbutenamide warhead with NF764 and NF765 improved CTNNB1 binding and showed more potent loss of CTNNB1 HiBiT levels in cells. We also confirmed that both NF764 and NF765 lowered CTNNB1 protein levels in cells in a proteasome-dependent manner. We thus demonstrated that the potency of EN83 can be improved through medicinal chemistry.

### Some Conclusions

The disclosed covalent ligand degraders of CTNNB1 act through direct covalent targeting of three distinct cysteines in the armadillo repeat domains of CTNNB1—C466, C5299, and C619—to destabilize and degrade CTNNB1 in a proteasome-dependent manner. The mode of degradation is distinct from traditional modes of targeted protein degradation, such as Proteolysis Targeting Chimeras (PROTACs) and molecular glue degraders, and rather occurs through a thermodynamic destabilization of CTNNB1 resulting from direct covalent binding, leading to a destabilization-mediated degradation of CTNNB1¹⁶⁻¹⁸ This is analogous to our previous findings with another oncogenic transcription factor MYC, where we discovered a covalent ligand EN4 that irreversibly targeted C171 in MYC leading to a thermal destabilization and loss of MYC protein levels¹⁵. Many transcription factors have often eluded classical drug discovery efforts because of their lack of well-defined binding pockets for small-molecule binding and also because many of them possess large segments of unstructured regions. Covalent targeting of ligandable cysteines within transcription factors causing their destabilization and degradation provides a useful strategy for pharmacologically tackling this challenging class of targets.

### Cell Culture

CTNNB1 HiBiT cells were commercially purchased through Promega (CS302340). HEK293, COLO-201, HT29, and SW480 were obtained from UC Berkeley's Biosciences Divisional Services Cell Culture Facility. HiBiT, HEK, and HT29 were cultured in DMEM and COLO-201 cells were cultured in RPMI base media. Both media types contained 10% (v/v) fetal bovine serum (FBS), were supplemented with 1% glutamine, and were maintained at 37° C. with 5% CO₂.

### Covalent Ligand Library and Synthesis of Other Compounds

Covalent ligands starting with “EN” are commercially available from Enamine LLC. Synthesis of other compounds are described herein.

Covalent Ligand Screen with CTNNB1 HiBiT Cell Line

Covalent ligand screen and dose responses were conducted using Promega Nano-Glo HiBiT Lytic Detection System (N3040) and CellTiter-Glo 2.0 Assay (G9242). HiBiT cells were seeded into 96-well plates (Corning 3917) at 35,000 cells per 100 μL of media and were left overnight to adhere. Cells were treated with 25 μL of media containing 1:125 dilution from a 1000×DMSO compound stock and treated for either 6 hrs, 12 hrs, or 24 hrs. The lytic detection system recipe was followed per Promega's suggestion. 125 μL of CTG or Lytic detection system reagents were added to each well. To assess the cells in the supernatant, half of the plates had the media removed prior to the addition of CTG or lytic detection system reagents. Plates were rocked for 15 minutes prior to their luminescence readout on the Tecan Spark Plate reader (30086376).

### Western Blotting

Pelleted cells are lysed with CST buffer containing protease inhibitor cocktail (Pierce A32955). Samples were centrifuged at 20,000 g for 20 min at 4° C. to remove cell debris. Samples' protein content was normalized to run 30 μg per well. Samples were then boiled for 8 min at 90° C. after addition of 4×reducing Laemmli SDS sample loading buffer (Alfa Aesar) and ran on precast 4-20% Criterion TGX gels (Bio-Rad).

Antibodies to CTNNB1 (Cell Signaling Technology, 8814S), GAPDH (Proteintech Group Inc., 60004-1-Ig or Cell Signaling Technology, 14C10), and DDDDK tag (Abcam, ab205606) were diluted per recommended manufacturers' procedures. Proteins were resolved by SDS/PAGE and transferred to nitrocellulose membranes using the BioRad system (1704271 and 1704150). Blots were blocked with 5% BSA in Tris-buffered saline containing Tween 20 (TBST) solution for 1 hour at room temperature, washed in TBST, and probed with primary antibody diluted in recommended diluent per manufacturer overnight at 4° C. Following washes with TBST, the blots were incubated in the dark with secondary antibodies purchased from Ly-Cor and used at 1:10,000 dilution in 5% BSA in TBST at room temperature. Blots were visualized using an Odyssey Li-Cor scanner after additional washes. If additional primary antibody incubations were required the membrane was stripped using ReBlot Plus Strong Antibody Stripping Solution (EMD Millipore, 2504), washed and blocked again before being reincubated with primary antibody.

Covalent Ligand Screen and Gel-Based ABPP with CTNNB1 Pure Protein

CTNNB1 pure protein (0.1 μg/25 μL in PBS) was treated with either DMSO vehicle or covalent ligand at 37° C. for 30 min, and subsequently treated with 0.1 μM IA-Rhodamine (Setareh Biotech) for 1 h at RT in the dark. The reaction was stopped by addition of 4×reducing Laemmli SDS sample loading buffer (Alfa Aesar). After boiling at 95° C. for 5 min, the samples were separated on precast 4-20% Criterion TGX gels (Bio-Rad) and were analyzed by in-gel fluorescence using a ChemiDoc MP (Bio-Rad).

### NF686 Probe Labeling on CTNNB1 NV03 Pure Protein

CTNNB1 pure protein (0.2 μg/50 μL in PBS) was treated with either DMSO vehicle or NF686 at 37° C. for 30 min. Click reagents Azide-Fluor 545 (Click Chemistry Tools, Inc. AZ109-5), Copper (II) Sulfate, and TBTA (TCI Chemicals, T2993) were added to have a final concentration of 21.8 μM, 873.4 μM, and 47.2 μg/mL, respectively, for 1 hr at RT in the dark. The reaction was stopped by addition of 4×reducing Laemmli SDS sample loading buffer (Alfa Aesar). After boiling samples at 95° C. for 5 min, the samples were separated on precast 4-20% Criterion TGX gels (Bio-Rad). Probe-labeled proteins were analyzed by in-gel fluorescence using a ChemiDoc MP (Bio-Rad).

### Synthetic Methods and Characterization

All chemical reactions were carried out under a nitrogen atmosphere with dry solvents under anhydrous conditions, unless otherwise noted. Reagents were purchased at the highest commercial quality and used without further purification, unless otherwise stated. Room temperature is defined as between 21-25° C. Reactions were stirred magnetically and monitored by thin layer chromatography (TLC) using TLC plates pre-coated with silica gel 60 F254 on aluminium (Merck KGaA). Detection was by UV (254 nm and 365 nm) or chemical stain (KMnO₄, ninhydrin, iodine). Solvents were removed in vacuo using a Buchi R-300 Rotavapor (equipped with an I-300 Pro Interface, B-300 Base Heating Bath, Welch 2037B-01 DryFast pump, and VWR AD15R-40-V11B Circulating Bath). Solvents for silica gel chromatography were used as supplied by Sigma-Aldrich. Automated flash chromatography was performed on a Biotage Isolera instrument, equipped with a UV detector. Chromatograms were recorded at 254 and 280 nm. High-resolution mass spectra (FIRMS) were obtained on a Q Exactive Plus mass spectrometer (Thermo Fisher Scientific). ¹H and ¹³C Nuclear Magnetic Resonance (NMR) spectra were recorded on BRUKER AV spectrometer operating at 600 MHz for ¹H and at 150 MHz for ¹³C NMR. Measurements were carried out at ambient temperature. Chemical shifts (δ) are reported in ppm with the residual solvent signal as internal standard (chloroform at 7.26 and 77.2 ppm for ¹H NMR and ¹³C NMR, respectively). The multiplicity of each signal is indicated as s=singlet, d=doublet, t=triplet, q=quartet, quin=quintet, m=multiplet (i.e. complex peak obtained due to overlap), app=apparent or a combination of these. Coupling constants (J) are reported in Hertz (Hz). ¹³C NMR spectra were recorded with broadband ¹H decoupling.

### 1-(2-(2,5-dimethoxyphenyl)pyrrolidin-1-yl)prop-2-en-1-one (NF601)

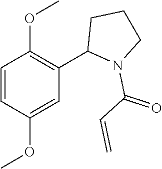 [SMILES: C=CC(=O)N1CCCC1c1cc(OC)ccc1OC]

2-(2,5-Dimethoxyphenyl)pyrrolidine (100 mg, 0.483 mmol) was dissolved in DMF (10 mL). To this was added acryloyl chloride (94.0 μL, 1.16 mmol), followed by NEt₃ (241 μL, 1.73 mmol) and the resultant mixture was stirred at RT for 18 h. It was then concentrated in vacuo and purification by column chromatography (gradient elution from CH₂Cl₂ to 70% diethyl ether in CH₂Cl₂) afforded the target product as a clear oil (89.0 mg, 0.341 mmol, 71%).

¹H NMR (CDCl₃, 600 MHz) δ_{H} 6.82-6.67 (m, 2H), 6.59-6.52 (m, 1H), 6.37-6.29 (m, 1H), 6.12-6.07 (m, 1H), 5.99-5.28 (m, 2H), 3.85-3.81 (m, 4H), 3.74-3.68 (m, 4H), 2.35-2.19 (m, 1H), 1.97-1.80 (m, 3H); ¹³C NMR (CDCl₃, 150 MHz) δ_{C} 165.2, 164.1, 153.7, 153.4, 150.5, 149.8, 132.6 132.3, 128.9, 128.7, 127.9, 127.3, 113.2, 113.0, 112.0, 111.4, 111.1, 110.6, 56.5, 56.4, 55.9, 55.7, 55.7, 55.6, 47.7, 47.0, 34.1, 32.2, 32.7, 21.7; HRMS (ES+) calcd for C₁₅H₁₉NO₃Na [M+Na]⁺ 284.1263, observed 284.1252.

### 1-(2-(2,5-dimethoxyphenyl)pyrrolidin-1-yl)propan-1-one (NF602)

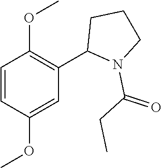 [SMILES: CCC(=O)N1CCCC1c1cc(OC)ccc1OC]

2-(2,5-dimethoxyphenyl)pyrrolidine (100 mg, 0.483 mmol) was dissolved in DMF (10 mL). To this was added propionyl chloride (101 μL, 1.16 mmol), followed by NEt₃ (241 μL, 1.73 mmol) and the resultant mixture was stirred at RT for 18 h. It was then concentrated in vacuo and purification by column chromatography (gradient elution from CH₂Cl₂ to 10% MeOH in CH₂Cl₂) afforded the target product as a clear oil (119 mg, 0.452 mmol, 94%).

¹H NMR (CDCl₃, 600 MHz) δ_{H} 6.81-6.65 (m, 2H), 6.58-6.47 (m, 1H), 5.42-5.17 (m, 1H), 3.80 (d, 3H, J=16.5 Hz), 3.77-3.73 (m, 1H), 3.72 (d, 3H, J=3.8 Hz), 3.68-3.54 (m, 1H), 2.40-2.56 (m, 1H), 2.22-2.12 (m, 1H), 1.99-1.74 (m, 4H), 1.15 (t, 1H, J=7.5 Hz), 1.00 (t, 2H, J=7.5 Hz); ¹³C NMR (CDCl₃, 150 MHz) δ_{C} 173.3, 172.1, 153.7, 153.4, 150.5, 150.1, 132.8, 132.6, 112.9, 112.7, 111.8, 111.4, 111.2, 110.5, 56.5, 56.0, 55.9, 55.7, 55.7, 55.5, 47.6, 46.7, 34.1, 32.2, 28.0, 27.4, 23.6, 21.7, 9.2, 9.0; HRMS (ES+) calcd for C₁₅H₂₁NO₃Na [M+Na]⁺ 286.1419, observed 286.1408.

### 3-(1-(2-chloroacetyl)pyrrolidin-2-yl)-4-methoxybenzoic acid (1)

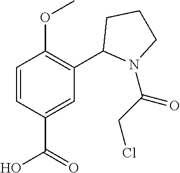 [SMILES: COc1ccc(C(=O)O)cc1C1CCCN1C(=O)CCl]

(S)-4-Methoxy-3-(pyrrolidin-2-yl)benzoic acid hydrochloride (100 mg, 0.388 mmol) was dissolved in DMF (8 mL). To this was added chloroacetyl chloride (37.0 μL, 0.465 mmol) at 0° C., followed by NEt₃ (130 μL, 0.933 mmol) and the resultant mixture was stirred at RT for 18 h. It was then concentrated in vacuo and purification by column chromatography (gradient elution from CH₂Cl₂ with 1% AcOH to 10% MeOH in CH₂Cl₂ with 1% AcOH) afforded the target product as an off white solid (112 mg, 0.377 mmol, 97%).

¹H NMR (CDCl₃, 600 MHz) δ_{H} 8.09-7.98 (m, 1H), 7.76-7.69 (m, 1H), 6.99-6.91 (m, 1H), 5.47-5.35 (m, 1H), 4.18-4.11 (m, 1H), 4.00-3.84 (m, 4H), 3.84 3.84-3.70 (m, 2H), 2.41-2.25 (m, 1H), 2.03-1.84 (m, 3H); ¹³C NMR (CDCl₃, 150 MHz) δ_{C} 171.2, 170.6, 165.8, 165.4, 160.5, 160.1, 131.9, 131.2, 130.4, 130.2, 127.8, 127.3, 112.3, 121.4, 110.4, 110.1, 56.7, 55.6, 55.9, 55.7, 47.8, 47.8, 42.3, 41.8, 34.2, 32.0, 23.8, 21.7.

### (S)-3-(1-(2-chloroacetyl)pyrrolidin-2-yl)-4-methoxy-N-(pent-4-yn-1-yl)benzamide (NF686)

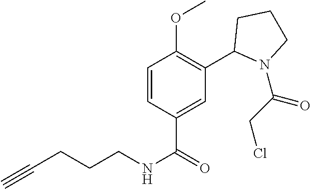 [SMILES: C#CCCCNC(=O)c1ccc(OC)c(C2CCCN2C(=O)CCl)c1]

To 1 (50.0 mg, 0.168 mmol) in CH₂Cl₂ (5 mL) was added T3P (109 μL, 0.184 mmol, 50% solution in EtOAc), followed by NEt₃ (86.0 μL, 0.617 mmol) and 4-pentyn-1-amine (16.0 μL, 0.153 mmol). The resultant mixture was stirred at RT for 18 h, It was then concentrated in vacuo and purification by column chromatography (gradient elution from CH₂Cl₂ to 10% MeOH in CH₂Cl₂) afforded the target product as a clear oil (51.0 mg, 0.141 mmol, 92%).

¹H NMR (CDCl₃, 600 MHz) δ_{H} 7.87-7.65 (m, 1H), 7.51-7.39 (m, 1H), 6.98-6.87 (m, 1.5H), 6.44 (br s, 0.5H), 5.42-5.33 (m, 1H), 4.17-4.08 (m, 1H), 3.97-3.86 (m, 4H), 3.85-3.67 (m, 2H), 3.55-3.52 (m, 2H), 2.41-2.24 (m, 3H), 2.08-2.02 (m, 1H), 2.01-1.96 (m, 1H), 1.94-1.81 (m, 4H); ¹³C NMR (CDCl₃, 150 MHz) δ_{C} 167.5, 166.7, 165.7, 164.9, 158.7, 158.2, 130.0, 129.0, 127.4, 126.6, 124.1, 124.0, 110.5, 110.3, 84.0, 83.7, 69.3, 69.2, 56.7, 56.6, 55.8, 55.6, 47.9, 47.8, 42.5, 42.2, 39.4, 39.2, 34.4, 32.0, 28.1, 28.1, 23.9, 21.8, 16.4, 16.3; HRMS (ES+) calcd for C₁₉H₂₃ClN₂O₃Na [M+Na]⁺ 385.1295, observed 385.1286.

### tert-butyl 2-(2-methoxy-5-(trifluoromethyl)phenyl)-1H-pyrrole-1-carboxylate (2)

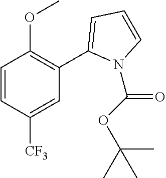 [SMILES: COc1ccc(C(F)(F)F)cc1-c1cccn1C(=O)OC(C)(C)C]

A mixture of 3-bromo-4-methoxybenzotrifluoride (1.02 g, 4.00 mmol), 1-Boc-pyrrole-2-boronic acid pinacol ester (1.76 g, 6.00 mmol) and Cs₂CO₃ (3.90 g, 12.0 mmol) in dioxane (16 mL)/H₂O (4 mL) was degassed for 20 min. XPhos Pd G4 (516 mg, 0.60 mmol) was then added and the mixture was further degassed for 10 min before it was heated to 100° C. for 16 h. Subsequent concentration in vacuo and purification by column chromatography (gradient elution from hexanes to 10% diethyl ether in hexanes) afforded the target product as a pale yellow oil (947 mg, 2.78 mmol, 70%).

¹H NMR (CDCl₃, 600 MHz) δ_{H} 7.65-7.63 (m, 1H), 7.60 (d, 1H, J=2.3 Hz), 7.44 (dd, 1H, J=3.3, 1.8 Hz), 6.98 (d, 1H, J=8.6 Hz), 6.32 (t, 1H, J=3.3 Hz), 6.25 (dd, 1H, J=3.3, 1.8 Hz), 3.86 (s, 3H), 1.41 (s, 9H); ¹³C NMR (CDCl₃, 150 MHz) δ_{C} 159.9, 149.2, 129.6, 127.4 (q, J=3.6 Hz), 126.3 (q, J=3.9 Hz), 125.4, 124.9, 123.6, 122.7, 122.4, 122.4, 114.6, 110.5, 109.8, 83.2, 55.6, 27.5; HRMS (ES+) calcd for C₁₇H₁₈F₃NO₃Na [M+Na]⁺ 364.1136, observed 364.1125.

### 2-(2-methoxy-5-(trifluoromethyl)phenyl)pyrrolidine (3)

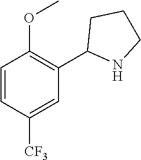 [SMILES: COc1ccc(C(F)(F)F)cc1C1CCCN1]

To 2 (100 mg, 0.293 mmol) in MeOH (10 mL) was added Rh/Al₂O₃ (30.0 mg, 5 wt. %). The resultant mixture was stirred under a H₂ atmosphere for 5 h, after which time it was filtered through a pad of celite and concentrated in vacuo. Conversion to the target pyrrolidine was confirmed by HRMS (ES+, calcd for C₁₇H₂₂F₃NO₃Na [2M+Na]⁺ 713.3003, observed 713.2988). The resultant product (101 mg, 0.293 mmol) was then dissolved in 4M HCl in dioxane (6 mL) and stirred at RT for 2 h. It was then concentrated in vacuo and used in the next steps without any further purification.

### 2-chloro-1-(2-(2-methoxy-5-(trifluoromethyl)phenyl)pyrrolidin-1-yl)ethan-1-one (NF740)

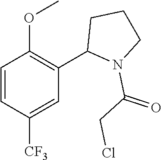 [SMILES: COc1ccc(C(F)(F)F)cc1C1CCCN1C(=O)CCl]

Compound 3 (105 mg, 0.304 mmol) was dissolved in DMF (5 mL). To this was added chloroacetyl chloride (36.0 μL, 0.453 mmol) at 0° C., followed by NEt₃ (127 μL, 0.911 mmol) and the resultant mixture was stirred at RT for 18 h. It was then concentrated in vacuo and purification by column chromatography (gradient elution from CH₂Cl₂ to 30% diethyl ether in CH₂Cl₂) afforded the target product as an off white crystalline solid (65.0 mg, 0.202 mmol, 69%).

¹H NMR (CDCl₃, 600 MHz) δ_{H} 7.58-7.46 (m, 1H), 7.23-7.20 (m, 1H), 7.00-6.92 (m, 1H), 5.45-5.36 (m, 1H), 4.14-4.06 (m, 1H), 3.96-3.82 (m, 4H), 3.82-3.67 (m, 1H), 2.43-2.24 (m, 1H), 2.04-1.78 (m, 3H); ¹³C NMR (CDCl₃, 150 MHz) δ_{C} 165.6, 165.0, 158.6, 58.3, 131.2, 130.9, 127.2, 127.2, 126.8, 126.4 (q, J=3.9 Hz), 125.5 (q, J=3.9 Hz), 125.4, 125.0, 123.6, 123.4, 123.2, 123.0, 122.7 (q, J=3.6 Hz), 122.6, 122.4, 122.3 (q, J=3.7 Hz), 122.1, 121.8, 1221.4, 110.6, 110.4, 56.6, 56.4, 55.9, 55.6, 47.8, 47.7, 42.1, 41.8, 34.1, 32.0, 23.9, 21.7; HRMS (ES+) calcd for C₁₄H₁₅ClF₃NO₂Na [2M+Na]⁺ 665.1384, observed 665.1375.

### (E)-1-(2-(2-methoxy-5-(trifluoromethyl)phenyl)pyrrolidin-1-yl)-4-phenylbut-2-ene-1,4-dione (NF765)

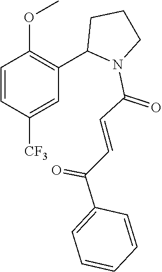 [SMILES: COc1ccc(C(F)(F)F)cc1C1CCCN1C(=O)/C=C/C(=O)c1ccccc1]

To trans-3-benzoylacrylic acid (42.0 mg, 0.238 mmol) in DMF (3 mL) was added HATU (130 mg, 0.342 mmol) and DIPEA (125 μL, 0.718 mmol), followed by 3 (45.0 mg, 0.184 mmol) in DMF (1 mL). The resultant mixture was stirred at RT for 18 h. It was then concentrated in vacuo and purified by column chromatography (gradient elution from hexanes to 30% diethyl ether in hexanes) to afford the target product as a yellow oil (38.0 mg, 0.094 mmol, 51%).

¹H NMR (CDCl₃, 600 MHz) δ_{H} 8.06-7.99 (m, 1H) 7.90-7.87 (m, 2H), 7.62-7.55 (m, 1H), 7.53-7.43 (m, 3H), 7.24-7.18 (m, 1H), 6.98-6.89 (m, 2H), 5.57-5.47 (m, 1H), 4.04-3.87 (m, 4H), 3.86-3.80 (m, 1H), 2.44-2.29 (m, 1H), 2.04-1.85 (m, 3H); ¹³C NMR (CDCl₃, 150 MHz) δ_{C} 189.9, 189.8, 164.0, 163.0, 158.6, 158.1, 137.0, 136.9, 136.9, 134.4, 134.0, 133.9, 133.7, 133.5, 133.1, 133.0, 131.9, 131.1, 128.9 (app q, J=7.1 Hz), 128.8 (app q, J=7.7 Hz), 128.7, 128.7, 126.3 (app q, J=3.0), 125.6 (app q, J=3.5 Hz), 123.2 (app q, J=3.4 Hz), 123.1, 122.9, 122.7, 122.5 (app q, J=4.2 Hz), 110.5, 110.5, 56.8, 56.4, 55.9, 55.7, 48.0, 47.5, 34.0, 32.1, 23.9, 21.8; HRMS (ES+) calcd for C₂₂H₂₀F₃NO₃Na [M+Na]⁺ 426.1293 observed 426.1283.

### tert-butyl 2-(5-methoxy-2-(trifluoromethyl)phenyl)-1H-pyrrole-1-carboxylate (4)

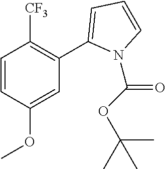 [SMILES: COc1ccc(C(F)(F)F)c(-c2cccn2C(=O)OC(C)(C)C)c1]

A mixture of 2-bromo-4-methoxy-1-(trifluoromethyl)benzene (692 mg, 2.71 mmol), 1-Boc-pyrrole-2-boronic acid pinacol ester (1.20 g, 4.09 mmol) and Cs₂CO₃ (2.60 g, 7.98 mmol) in dioxane (10.8 mL)/H₂O (2.7 mL) was degassed for 20 min. XPhos Pd G4 (348 mg, 0.40 mmol) was then added and the mixture was further degassed for 10 min before it was heated to 100° C. for 16 h. Subsequent concentration in vacuo and purification by column chromatography (gradient elution from hexanes to 10% diethyl ether in hexanes) afforded the target product as a pale yellow oil (723 mg, 2.12 mmol, 78%).

¹H NMR (CDCl₃, 600 MHz) δ_{H} 7.64 (d, 1H, J=8.8 Hz), 7.44 (dd, 1H, J=3.4, 1.8 Hz), 6.99 (ddd, 1H, J=8.8, 2.7, 1.0 Hz), 6.92 (d, 1H, J=2.6 Hz), 6.28 (t, 1H, J=3.3 Hz), 6.25-6.14 (m, 1H), 3.87 (s, 3H), 1.29 (s, 9H); ¹³C NMR (CDCl₃, 150 MHz) δ_{C} 161.1, 148.9, 135.6 (q, J 2.1 Hz), 129.4, 127.3 (q, J=5.0 Hz), 125.2, 123.4, 122.5, 122.3, 122.0, 121.7, 118.0, 115.0, 113.1, 110.3, 83.3, 55.5, 27.4; HRMS (ES+) calcd for C₁₇H₁₈F₃NO₃Na [M+Na]⁺ 364.1136, observed 364.1124.

### 2-(5-methoxy-2-(trifluoromethyl)phenyl)pyrrolidine (5)

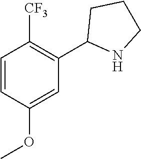 [SMILES: COc1ccc(C(F)(F)F)c(C2CCCN2)c1]

To 4 (100 mg, 0.293 mmol) in MeOH (10 mL) was added Rh/Al₂O₃ (30.0 mg, 5 wt. %). The resultant mixture was stirred under a H₂ atmosphere for 1.5 h, after which time it was filtered through a pad of celite and concentrated in vacuo. Conversion to the target pyrrolidine was confirmed by HRMS (ES+, calcd for C₁₇H₂₂F₃NO₃Na [M+Na]⁺ 368.1449, observed 368.1439). The resultant product (101 mg, 0.293 mmol) was then dissolved in 4M HCl in dioxane (6 mL) and stirred at RT for 2 h. It was then concentrated in vacuo and used in the next steps without any further purification.

### 2-chloro-1-(2-(5-methoxy-2-(trifluoromethyl)phenyl)pyrrolidin-1-yl)ethan-1-one (NF741)

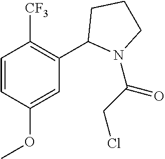 [SMILES: COc1ccc(C(F)(F)F)c(C2CCCN2C(=O)CCl)c1]

Compound 5 was dissolved in DMF (5 mL). To this was added chloroacetyl chloride (36.0 μL, 0.453 mmol) at 0° C., followed by NEt₃ (127 μL, 0.911 mmol) and the resultant mixture was stirred at RT for 18 h. It was then concentrated in vacuo and purification by column chromatography (gradient elution from CH₂Cl₂ to 30% diethyl ether in CH₂Cl₂) afforded the target product as a white solid (53.0 mg, 0.165 mmol, 56%).

¹H NMR (CDCl₃, 600 MHz) δ_{H} 7.66-7.58 (m, 1H), 6.89-6.72 (m, 2H), 5.46-5.34 (m, 1H), 4.14-4.03 (m, 1H), 4.00-3.87 (m, 1H), 3.88-3.59 (m, 5H), 2.56-2.36 (m, 1H), 2.16-1.79 (m, 3H); ¹³C NMR (CDCl₃, 150 MHz) δ_{C} 165.6, 164.7, 162.6, 162.2, 143.9, 143.7, 128.7 (q, J=5.9 Hz), 128.3 (q, J=5.9 Hz), 127.4, 127.2, 125.6, 125.4, 123.8, 123.6, 122.0, 121.8, 119.6, 119.4, 119.2, 119.2, 119.0, 118.8, 118.6, 113.2, 111.7, 111.5, 110.9, 58.1 (q, J=2.4 Hz), 57.8 (q, J=2.2 Hz), 55.6, 55.4, 48.4, 48.3, 42.4, 42.0, 36.1, 34.0, 24.1, 21.5; HRMS (ES+) calcd for C₁₄H₁₅ClF₃NO₂H [M+H]⁺ 322.0822, observed 322.0814.

### (E)-1-(2-(5-methoxy-2-(trifluoromethyl)phenyl)pyrrolidin-1-yl)-4-phenylbut-2-ene-1,4-dione (NF764)

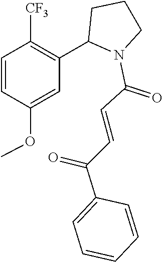 [SMILES: COc1ccc(C(F)(F)F)c(C2CCCN2C(=O)/C=C/C(=O)c2ccccc2)c1]

To trans-3-Benzoylacrylic acid (42.0 mg, 0.238 mmol) in DMF (3 mL) was added HATU (130 mg, 0.342 mmol) and DIPEA (125 μL, 0.718 mmol), followed by 5 (45.0 mg, 0.184 mmol) in DMF (1 mL). The resultant mixture was stirred at RT for 18 h. It was then concentrated in vacuo and purified by column chromatography (gradient elution from CH₂Cl₂ to 30% diethyl ether in CH₂Cl₂) to afford the target product as an orange oil (49.0 mg, 0.122 mmol, 66%).

¹H NMR (CDCl₃, 600 MHz) bx 8.04-8.01 (m, 1H), 7.86-7.79 (m, 2H), 7.64-7.60 (m, 1H), 7.58-7.42 (m, 3H), 6.92-6.90 (m, 1H), 6.85-6.72 (m, 2H), 5.57-5.48 (m, 1H), 4.06-3.86 (m, 2H), 3.82-3.81 (m, 3H), 2.57-2.42 (m, 1H), 2.20-1.85 (m, 3H); ¹³C NMR (CDCl₃, 150 MHz) be 189.8, 189.6, 164.0, 162.9, 162.5, 162.2, 114.4, 144.2, 136.9, 136.9, 134.6, 134.4, 133.7, 133.4, 133.0, 132.7, 128.8, 128.8, 128.7, 128.7, 128.3, 125.5, 123.7, 119.6, 119.4, 118.9, 118.7, 113.7, 112.6, 111.3, 110.3, 58.3 (app q, J=2.4 Hz), 58.1 (q, J=2.3 Hz), 55.5, 55.3, 48.4, 48.1, 36.0, 34.1, 24.0, 21.7; HRMS (ES+) calcd for C₂₂H₂₀F₃NO₃H [M+H]+ 404.1474, observed 404.1466.

### REFERENCES

1. Bugter, J. M., Fenderico, N. & Maurice, M. M. Mutations and mechanisms of WNT pathway tumour suppressors in cancer. Nat. Rev. Cancer 21, 5-21 (2021).
2. Anastas, J. N. & Moon, R. T. WNT signalling pathways as therapeutic targets in cancer. Nat. Rev. Cancer 13, 11-26 (2013).
3. Lecarpentier, Y., Schussler, O., Hebert, J.-L. & Vallee, A. Multiple Targets of the Canonical WNT/β-Catenin Signaling in Cancers. Front. Oncol. 9, 1248 (2019).
4. Doghman, M., Cazareth, J. & Lalli, E. The T cell factor/beta-catenin antagonist PKF115-584 inhibits proliferation of adrenocortical carcinoma cells. J. Clin. Endocrinol. Metab. 93, 3222-3225 (2008).
5. Dev, A., Vachher, M. & Prasad, C. P. β-catenin inhibitors in cancer therapeutics: intricacies and way forward. Bioengineered 14, 2251696.
6. Danieau, G. et al. ICG-001, an Inhibitor of the β-Catenin and cAMP Response Element-Binding Protein Dependent Gene Transcription, Decreases Proliferation but Enhances Migration of Osteosarcoma Cells. Pharm. Basel Switz. 14, 421 (2021).
7. Martins-Neves, S. R. et al. IWR-1, a tankyrase inhibitor, attenuates Wnt/β-catenin signaling in cancer stem-like cells and inhibits in vivo the growth of a subcutaneous human osteosarcoma xenograft. Cancer Lett. 414, 1-15 (2018).
8. Stakheev, D. et al. The WNT/β-catenin signaling inhibitor XAV939 enhances the elimination of LNCaP and PC-3 prostate cancer cells by prostate cancer patient lymphocytes in vitro. Sci. Rep. 9, 4761 (2019).
9. Hwang, S.-Y. et al. Direct Targeting of β-Catenin by a Small Molecule Stimulates Proteasomal Degradation and Suppresses Oncogenic Wnt/β-Catenin Signaling. Cell Rep. 16, 28-36 (2016).
10. Boike, L., Henning, N. J. & Nomura, D. K. Advances in covalent drug discovery. Nat. Rev. Drug Discov. 21, 881-898 (2022).
11. Liu, Y., Patricelli, M. P. & Cravatt, B. F. Activity-based protein profiling: the serine hydrolases. Proc. Natl. Acad. Sci. U.S.A 96, 14694-14699 (1999).
12. Backus, K. M. et al. Proteome-wide covalent ligand discovery in native biological systems. Nature 534, 570-574 (2016).
13. Weerapana, E., Simon, G. M. & Cravatt, B. F. Disparate proteome reactivity profiles of carbon electrophiles. Nat. Chem. Biol. 4, 405-407 (2008).
14. Spradlin, J. N., Zhang, E. & Nomura, D. K. Reimagining Druggability Using Chemoproteomic Platforms. Acc. Chem. Res. 54, 1801-1813 (2021).
15. Boike, L. et al. Discovery of a Functional Covalent Ligand Targeting an Intrinsically Disordered Cysteine within MYC. Cell Chem. Biol. 28, 4-13.e17 (2021).
16. Connelly, C. M., Boer, R. E., Moon, M. H., Gareiss, P. & Schneekloth, J. S. Discovery of Inhibitors of MicroRNA-21 Processing Using Small Molecule Microarrays. ACS Chem. Biol. 12, 435-443 (2017).
17. Winter, G. E. et al. DRUG DEVELOPMENT. Phthalimide conjugation as a strategy for in vivo target protein degradation. Science 348, 1376-1381 (2015).
18. Krönke, J. et al. Lenalidomide causes selective degradation of IKZF1 and IKZF3 in multiple myeloma cells. Science 343, 301-305 (2014).

### SAR Validated NF Compounds

We performed SAR validations demonstrating micromolar dose-response activity (modulation of CTNNB1 levels) profile retention across methoxy substitutions in the aromatic ring, and warhead swapping. Validate NF series compounds shown in Table 1.

TABLE 1
SAR validated NF compounds

| | |
|---|---|
| 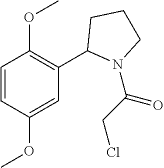 [SMILES: COc1ccc(OC)c(C2CCCN2C(=O)CCl)c1] | EN83 |
| 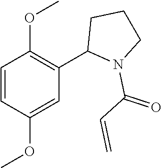 [SMILES: C=CC(=O)N1CCCC1c1cc(OC)ccc1OC] | NF601 |
| 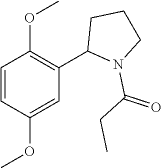 [SMILES: CCC(=O)N1CCCC1c1cc(OC)ccc1OC] | NF602 |
| 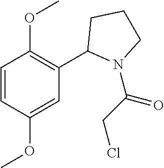 [SMILES: COc1ccc(OC)c(C2CCCN2C(=O)CCl)c1] | NF603 |
| 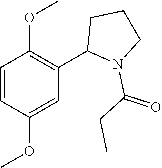 [SMILES: CCC(=O)N1CCCC1c1cc(OC)ccc1OC] | NF642 |
| 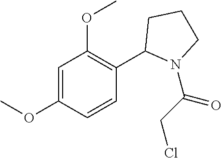 [SMILES: COc1ccc(C2CCCN2C(=O)CCl)c(OC)c1] | NF673 |
| 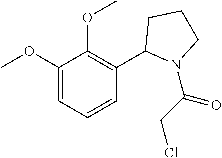 [SMILES: COc1cccc(C2CCCN2C(=O)CCl)c1OC] | NF692 |
| 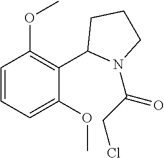 [SMILES: COc1cccc(OC)c1C1CCCN1C(=O)CCl] | NF693 |
| 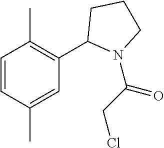 [SMILES: Cc1ccc(C)c(C2CCCN2C(=O)CCl)c1] | NF710 |
| 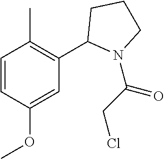 [SMILES: COc1ccc(C)c(C2CCCN2C(=O)CCl)c1] | NF711 |
| 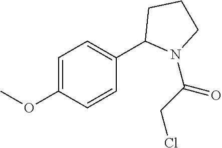 [SMILES: COc1ccc(C2CCCN2C(=O)CCl)cc1] | NF732 |
| 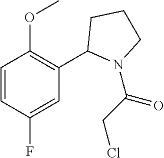 [SMILES: COc1ccc(F)cc1C1CCCN1C(=O)CCl] | NF733 |
| 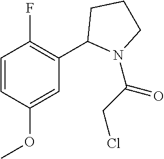 [SMILES: COc1ccc(F)c(C2CCCN2C(=O)CCl)c1] | NF734 |
| 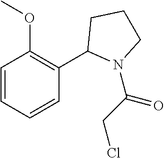 [SMILES: COc1ccccc1C1CCCN1C(=O)CCl] | NF735 |
| 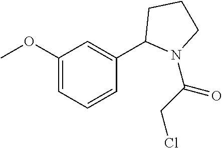 [SMILES: COc1cccc(C2CCCN2C(=O)CCl)c1] | NF736 |
| 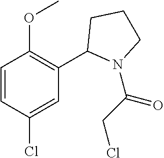 [SMILES: COc1ccc(Cl)cc1C1CCCN1C(=O)CCl] | NF742 |
| 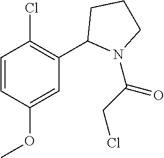 [SMILES: COc1ccc(Cl)c(C2CCCN2C(=O)CCl)c1] | NF745 |
| 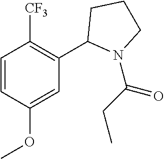 [SMILES: CCC(=O)N1CCCC1c1cc(OC)ccc1C(F)(F)F] | NF752 |
| 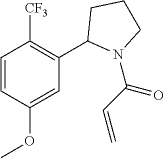 [SMILES: C=CC(=O)N1CCCC1c1cc(OC)ccc1C(F)(F)F] | NF753 |
| 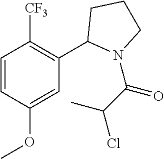 [SMILES: COc1ccc(C(F)(F)F)c(C2CCCN2C(=O)C(C)Cl)c1] | NF756 |
| 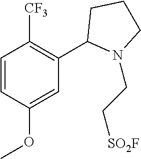 [SMILES: COc1ccc(C(F)(F)F)c(C2CCCN2CCS(=O)(=O)F)c1] | NF760 |
| 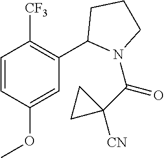 [SMILES: COc1ccc(C(F)(F)F)c(C2CCCN2C(=O)C2(C#N)CC2)c1] | NF761 |
| 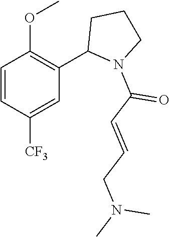 [SMILES: COc1ccc(C(F)(F)F)cc1C1CCCN1C(=O)/C=C/CN(C)C] | NF762 |
| 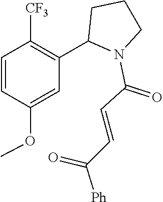 [SMILES: COc1ccc(C(F)(F)F)c(C2CCCN2C(=O)/C=C/C(=O)c2ccccc2)c1] | NF764 |
| 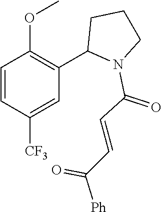 [SMILES: COc1ccc(C(F)(F)F)cc1C1CCCN1C(=O)/C=C/C(=O)c1ccccc1] | NF765 |
| 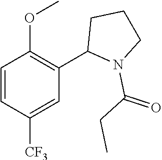 [SMILES: CCC(=O)N1CCCC1c1cc(C(F)(F)F)ccc1OC] | NF769 |
| 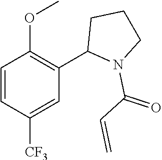 [SMILES: C=CC(=O)N1CCCC1c1cc(C(F)(F)F)ccc1OC] | NF770 |
| 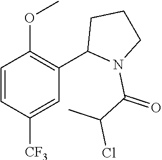 [SMILES: COc1ccc(C(F)(F)F)cc1C1CCCN1C(=O)C(C)Cl] | NF774 |
| 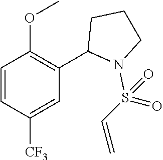 [SMILES: C=CS(=O)(=O)N1CCCC1c1cc(C(F)(F)F)ccc1OC] | NF775 |
| 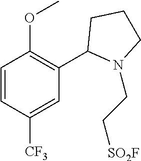 [SMILES: COc1ccc(C(F)(F)F)cc1C1CCCN1CCS(=O)(=O)F] | NF776 |
| 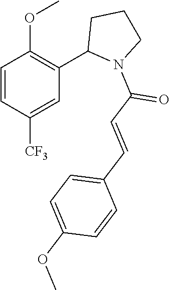 [SMILES: COc1ccc(/C=C/C(=O)N2CCCC2c2cc(C(F)(F)F)ccc2OC)cc1] | NF797 |
| 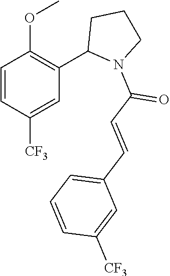 [SMILES: COc1ccc(C(F)(F)F)cc1C1CCCN1C(=O)/C=C/c1cccc(C(F)(F)F)c1] | NF798 |
| 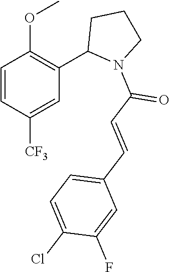 [SMILES: COc1ccc(C(F)(F)F)cc1C1CCCN1C(=O)/C=C/c1ccc(Cl)c(F)c1] | NF799 |
| 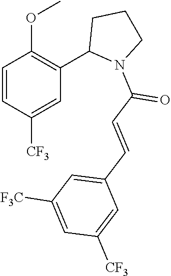 [SMILES: COc1ccc(C(F)(F)F)cc1C1CCCN1C(=O)/C=C/c1cc(C(F)(F)F)cc(C(F)(F)F)c1] | NF800 |
| 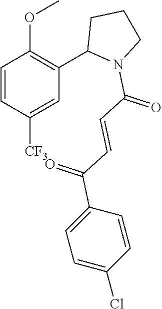 [SMILES: COc1ccc(C(F)(F)F)cc1C1CCCN1C(=O)/C=C/C(=O)c1ccc(Cl)cc1] | NF801 |
| 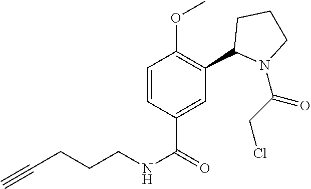 | NF686 |
| 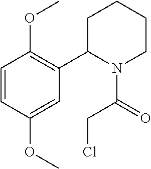 [SMILES: COc1ccc(OC)c(C2CCCCN2C(=O)CCl)c1] | NF688 |
| 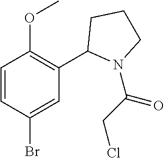 [SMILES: COc1ccc(Br)cc1C1CCCN1C(=O)CCl] | NF712 |
| 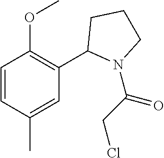 [SMILES: COc1ccc(C)cc1C1CCCN1C(=O)CCl] | NF720 |
| 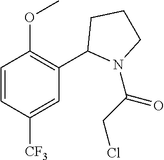 [SMILES: COc1ccc(C(F)(F)F)cc1C1CCCN1C(=O)CCl] | NF740 |
| 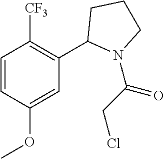 [SMILES: COc1ccc(C(F)(F)F)c(C2CCCN2C(=O)CCl)c1] | NF741 |
| 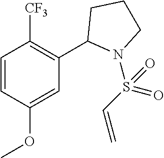 [SMILES: C=CS(=O)(=O)N1CCCC1c1cc(OC)ccc1C(F)(F)F] | NF757 |
| 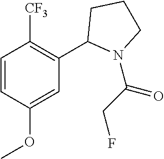 [SMILES: COc1ccc(C(F)(F)F)c(C2CCCN2C(=O)CF)c1] | NF759 |
| 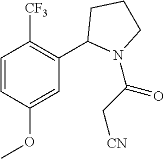 [SMILES: COc1ccc(C(F)(F)F)c(C2CCCN2C(=O)CC#N)c1] | NF767 |
| 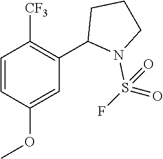 [SMILES: COc1ccc(C(F)(F)F)c(C2CCCN2S(=O)(=O)F)c1] | NF768 |
| 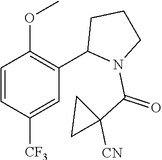 [SMILES: COc1ccc(C(F)(F)F)cc1C1CCCN1C(=O)C1(C#N)CC1] | NF777 |
| 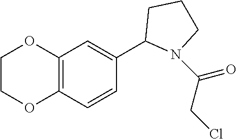 [SMILES: O=C(CCl)N1CCCC1c1ccc2c(c1)OCCO2] | NF781 |
| 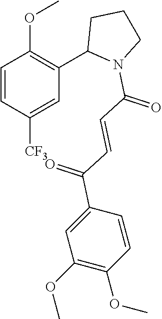 [SMILES: COc1ccc(C(=O)/C=C/C(=O)N2CCCC2c2cc(C(F)(F)F)ccc2OC)cc1OC] | NF802 |
| 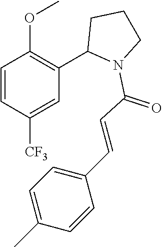 [SMILES: COc1ccc(C(F)(F)F)cc1C1CCCN1C(=O)/C=C/c1ccc(C)cc1] | NF803 |
| 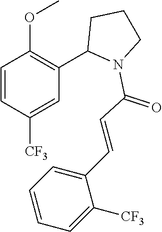 [SMILES: COc1ccc(C(F)(F)F)cc1C1CCCN1C(=O)/C=C/c1ccccc1C(F)(F)F] | NF804 |
| 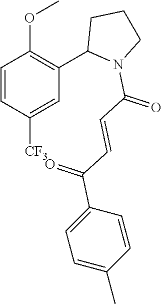 [SMILES: COc1ccc(C(F)(F)F)cc1C1CCCN1C(=O)/C=C/C(=O)c1ccc(C)cc1] | NF805 |
| 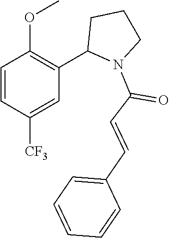 [SMILES: COc1ccc(C(F)(F)F)cc1C1CCCN1C(=O)/C=C/c1ccccc1] | NF806 |
| 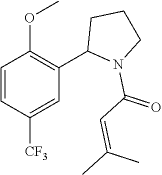 [SMILES: COc1ccc(C(F)(F)F)cc1C1CCCN1C(=O)C=C(C)C] | NF807 |
| 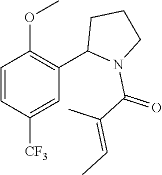 [SMILES: C/C=C(\C)C(=O)N1CCCC1c1cc(C(F)(F)F)ccc1OC] | NF808 |
| 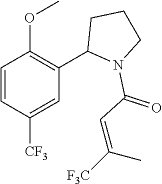 [SMILES: COc1ccc(C(F)(F)F)cc1C1CCCN1C(=O)/C=C(\C)C(F)(F)F] | NF809 |
| 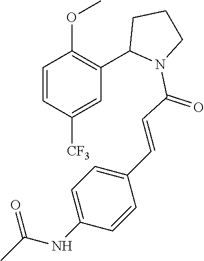 [SMILES: COc1ccc(C(F)(F)F)cc1C1CCCN1C(=O)/C=C/c1ccc(NC(C)=O)cc1] | NF810 |
| 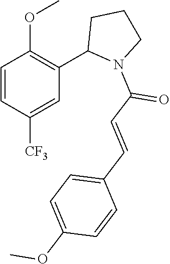 [SMILES: COc1ccc(/C=C/C(=O)N2CCCC2c2cc(C(F)(F)F)ccc2OC)cc1] | NF812 |
| 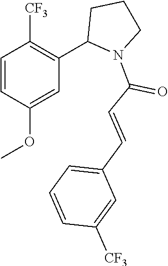 [SMILES: COc1ccc(C(F)(F)F)c(C2CCCN2C(=O)/C=C/c2cccc(C(F)(F)F)c2)c1] | NF813 |
| 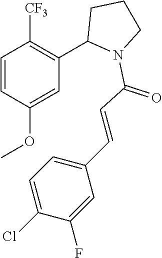 [SMILES: COc1ccc(C(F)(F)F)c(C2CCCN2C(=O)/C=C/c2ccc(Cl)c(F)c2)c1] | NF814 |
| 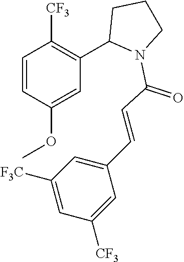 [SMILES: COc1ccc(C(F)(F)F)c(C2CCCN2C(=O)/C=C/c2cc(C(F)(F)F)cc(C(F)(F)F)c2)c1] | NF815 |
| 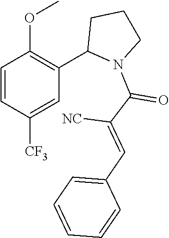 [SMILES: COc1ccc(C(F)(F)F)cc1C1CCCN1C(=O)/C(C#N)=C/c1ccccc1] | NF816 |
| 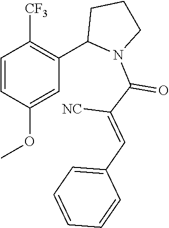 [SMILES: COc1ccc(C(F)(F)F)c(C2CCCN2C(=O)/C(C#N)=C/c2ccccc2)c1] | NF817 |
| 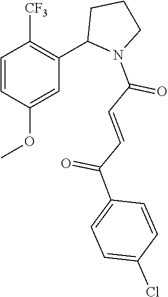 [SMILES: COc1ccc(C(F)(F)F)c(C2CCCN2C(=O)/C=C/C(=O)c2ccc(Cl)cc2)c1] | NF818 |
| 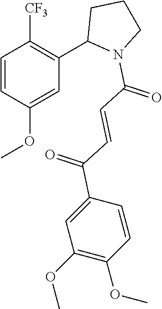 [SMILES: COc1ccc(C(F)(F)F)c(C2CCCN2C(=O)/C=C/C(=O)c2ccc(OC)c(OC)c2)c1] | NF819 |
| 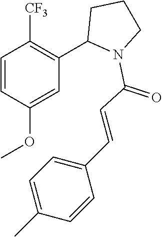 [SMILES: COc1ccc(C(F)(F)F)c(C2CCCN2C(=O)/C=C/c2ccc(C)cc2)c1] | NF820 |
| 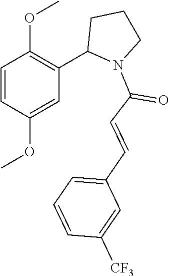 [SMILES: COc1ccc(OC)c(C2CCCN2C(=O)/C=C/c2cccc(C(F)(F)F)c2)c1] | NF821 |
| 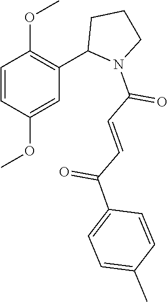 [SMILES: COc1ccc(OC)c(C2CCCN2C(=O)/C=C/C(=O)c2ccc(C)cc2)c1] | NF822 |

TABLE 2
Chloroacetamide warheads; see, Grabrijan et al.,
Dec 5, 2022, Eur J Med Chem, 243:114752.

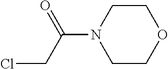

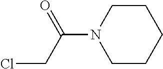

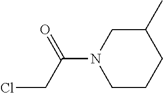

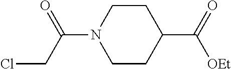 [SMILES: CCOC(=O)C1CCN(C(=O)CCl)CC1]

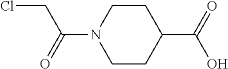 [SMILES: O=C(O)C1CCN(C(=O)CCl)CC1]

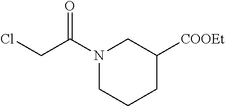 [SMILES: CCOC(=O)C1CCCN(C(=O)CCl)C1]

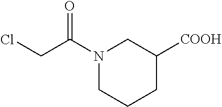 [SMILES: O=C(O)C1CCCN(C(=O)CCl)C1]

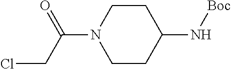 [SMILES: CC(C)(C)OC(=O)NC1CCN(C(=O)CCl)CC1]

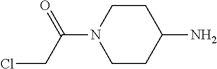 [SMILES: NC1CCN(C(=O)CCl)CC1]

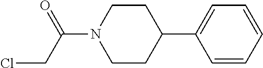 [SMILES: O=C(CCl)N1CCC(c2ccccc2)CC1]

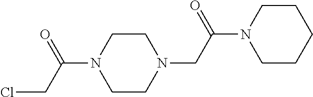 [SMILES: O=C(CCl)N1CCN(CC(=O)N2CCCCC2)CC1]

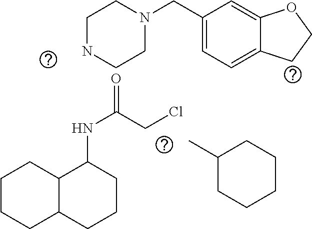

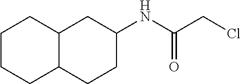 [SMILES: O=C(CCl)NC1CCC2CCCCC2C1]

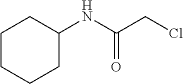 [SMILES: O=C(CCl)NC1CCCCC1]

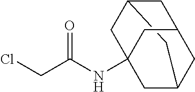 [SMILES: O=C(CCl)NC12CC3CC(CC(C3)C1)C2]

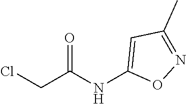

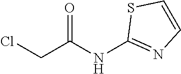

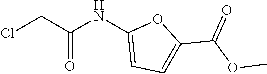

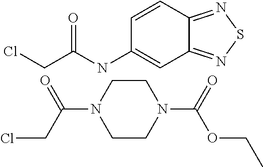

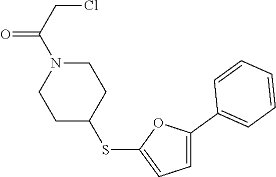 [SMILES: O=C(CCl)N1CCC(Sc2ccc(-c3ccccc3)o2)CC1]

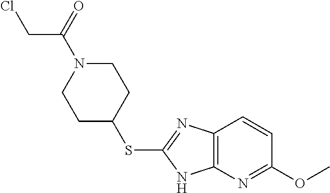 [SMILES: COc1ccc2nc(SC3CCN(C(=O)CCl)CC3)[nH]c2n1]

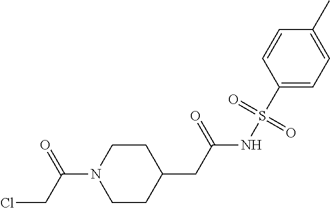 [SMILES: Cc1ccc(S(=O)(=O)NC(=O)CC2CCN(C(=O)CCl)CC2)cc1]

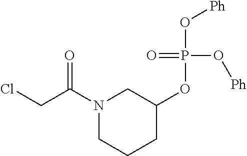 [SMILES: O=C(CCl)N1CCCC(OP(=O)(Oc2ccccc2)Oc2ccccc2)C1]

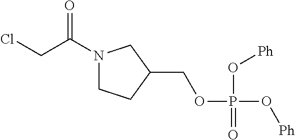 [SMILES: O=C(CCl)N1CCC(COP(=O)(Oc2ccccc2)Oc2ccccc2)C1]

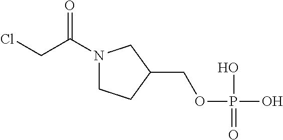 [SMILES: O=C(CCl)N1CCC(COP(=O)(O)O)C1]

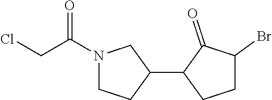 [SMILES: O=C1C(Br)CCC1C1CCN(C(=O)CCl)C1]

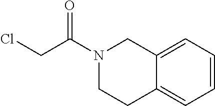 [SMILES: O=C(CCl)N1CCc2ccccc2C1]

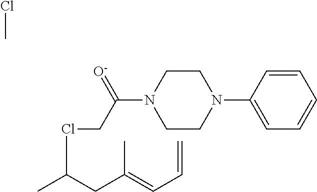

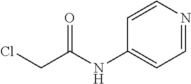 [SMILES: O=C(CCl)Nc1ccncc1]

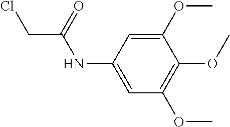 [SMILES: COc1cc(NC(=O)CCl)cc(OC)c1OC]

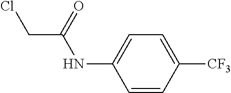 [SMILES: O=C(CCl)Nc1ccc(C(F)(F)F)cc1]

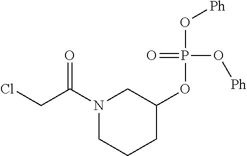 [SMILES: O=C(CCl)N1CCCC(OP(=O)(Oc2ccccc2)Oc2ccccc2)C1]

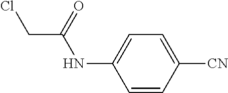 [SMILES: N#Cc1ccc(NC(=O)CCl)cc1]

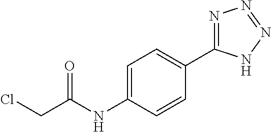 [SMILES: O=C(CCl)Nc1ccc(-c2nnn[nH]2)cc1]

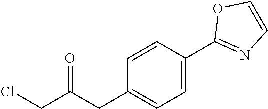 [SMILES: O=C(CCl)Cc1ccc(-c2ncco2)cc1]

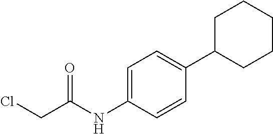 [SMILES: O=C(CCl)Nc1ccc(C2CCCCC2)cc1]

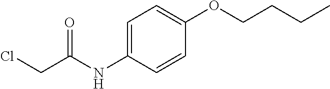 [SMILES: CCCCOc1ccc(NC(=O)CCl)cc1]

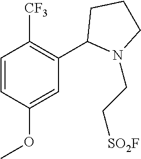 [SMILES: COc1ccc(C(F)(F)F)c(C2CCCN2CCS(=O)(=O)F)c1]

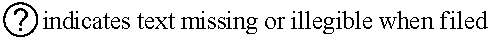

1. A compound of formula I: 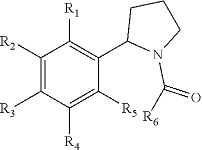
wherein
a) one of R1-R5 is methoxy, and the others are independently selected from H, halide, hydroxyl, C1-C4 alkyl, C1-C4 fluorinated alkyl, C1-C4 alkyloxy, C1-C4 fluorinated alkyloxy, and other substituents described herein, wherein two of the other R1-R5 may be joined in a 5 or 6 membered ring; and
b) NCOR6 is an amide-linked cysteine-reactive covalent ligand warhead,
including a pharmaceutically-acceptable salt thereof, and excluding EN83.

2. A compound of claim 1, wherein R2, R3 and R5 are H.

3. A compound of claim 1, wherein R2, R3 and R5 are H, R1 is CHF₃ and R4 is methoxy.

4. A compound of claim 1, wherein R2, R3 and R5 are H, R1 is CHF₃ and R4 is methoxy.

5. A compound of claim 1, wherein the warhead is selected from: EGFR inhibitors (such as gefitinib, erlotinib, fatinib, neratinib, dacomitinib, WZ4002, osimertinib, rociletinib), BTK inhibitors (such as ibrutinib, acalabrutinib, zanubrutinib), Janus kinase 3 (JAK3) inhibitors (such as ritlecitinib), fibroblast growth factor receptor 4 (FGFR4) inhibitors (such as fisogatinib, roblitinib), KRAS (G12C) inhibitors (such as sotorasib, adagrasib, JNJ-7469915), SARS-CoV-2 main protease inhibitors (such as nirmatrelvir, rupintrivir), proteasome inhibitors (such as bortezomib, carfilzomib, ixazomib, oprozomib), other boron-containing drugs (such as tavaborole, crisaborole, vaborbactam) and mutant-haemoglobin modulators (such as voxelotor).

6. A compound of claim 1, wherein the warhead is selected from: gefitinib, erlotinib, afatinib, neratinib, dacomitinib, WZ4002, osimertinib, rociletinib, ibrutinib, acalabrutinib, zanubrutinib, ritlecitinib, fisogatinib, roblitinib, sotorasib, adagrasib, JNJ-7469915, nirmatrelvir, rupintrivir, bortezomib, carfilzomib, ixazomib, oprozomib, tavaborole, crisaborole, vaborbactam and voxelotor.

7. A compound of 1, wherein the warhead comprises a chloroacetamide of Table 2.

8. A compound of 1, wherein COR6 is but-2-en-1-one or but-2-ene-1,4-dione linked phenyl or phenyl substituted with 1-5 substituents selected from: halide, hydroxyl, C1-C4 alkyl, C1-C4 fluorinated alkyl, C1-C4 alkyloxy, C1-C4 fluorinated alkyloxy, NRaRb, NHCORa, wherein Ra and Rb are independently H, C1-C4 alkyl, C1-C4 fluorinated alkyl, C1-C4 alkyloxy, C1-C4 fluorinated alkyloxy.

9. A compound of 1, wherein COR6 is but-2-en-1-one or but-2-ene-1,4-dione linked phenyl.

10. A compound of claim 1, of formula II: 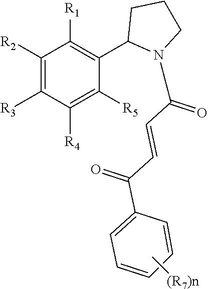
wherein:
a) one of R1-R5 is methoxy, and the others are independently selected from H, halide, hydroxyl, C1-C4 alkyl, C1-C4 fluorinated alkyl, C1-C4 alkyloxy, C1-C4 fluorinated alkyloxy, wherein two of the other R1-R5 may be joined in a 5 or 6 membered ring,
b) each R7 is independently selected from: H, halide, hydroxyl, C1-C4 alkyl, C1-C4 fluorinated alkyl, C1-C4 alkyloxy, C1-C4 fluorinated alkyloxy, NRaRb, NHCORa, or other substituents described herein, wherein Ra and Rb are independently H, C1-C4 alkyl, C1-C4 fluorinated alkyl, C1-C4 alkyloxy, C1-C4 fluorinated alkyloxy, and
c) n is 1-5.

11. A compound of claim 10, wherein R2, R3 and R5 are H.

12. A compound of claim 10, wherein R2, R3 and R5 are H, R1 is CHF₃ and R4 is methoxy.

13. A compound of claim 10, wherein R2, R3 and R5 are H, R1 is CHF₃ and R4 is methoxy.

14. A compound of claim 10, wherein each R7 is H.

15. A compound of claim 1, that is an NF compound of Table 1.

16. A compound of claim 1, that is NF764 of Table 1, comprising structure: 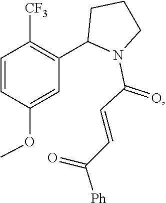 [SMILES: COc1ccc(C(F)(F)F)c(C2CCCN2C(=O)/C=C/C(=O)c2ccccc2)c1]
that is NF765 of Table 1, comprising structure:

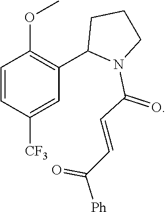 [SMILES: COc1ccc(C(F)(F)F)cc1C1CCCN1C(=O)/C=C/C(=O)c1ccccc1]

17. A protein comprising a cysteine covalently modified with a compound of claim 1,
optionally, comprising an oncogenic transcription factor, selected from: β-catenin/Wnt, NF-κB, AP1. STAT3, HIF-1, MYC, ETS1 and E2F, or
optionally, comprising a modified β-catenin (CTNNB1) comprising a covalent modification of C439, C466, C520 or C619.

18. A pharmaceutical composition comprising a compound of claim 1, and a pharmaceutically-acceptable excipient, in unit dosage or packaging.

19. A method of modifying a protein, comprising contacting the protein with a compound of claim 1, under conditions wherein a cysteine residue of the protein is covalently modified by the compound,
optionally, wherein the protein is selected from Wnt/β-catenin (CTNNB1), β-NF-κB, AP1. STAT3, HIF-1, MYC, ETS1, E2F; or
optionally, comprising modifying a protein comprising a β-catenin (CTNNB1), comprising covalently modifying C439, C466, C520 or C619.

20. A therapeutic method comprising administering to a person in need thereof a compound of claim 1,
optionally, to treat a cancer in the person, the cancer selected from: breast cancer, prostate cancer, colon cancer, multiple myeloma, liver cancer, ovarian cancer, lung cancer, acute myeloid leukemia, renal cancer, hepatocellular carcinoma, melanoma, colorectal cancer, bladder cancer, lung adenocarcinoma, pancreatic cancer, glioblastoma or anaplastic glioma, small-cell lung cancer, or other cancers associated with an oncogenic transcription factor,
optionally, to inhibit an oncogenic transcription factor in the person, such as: β-catenin/Wnt, NF-κB, AP1. STAT3, HIF-1, MYC, ETS1, E2F.
